(19) United States
(12) Patent Application Publication
(10) Pub. No.: US 2006/0180737 A1
(43) Pub. Date: Aug. 17, 2006

(54) System and method for detecting a label edge

(75) Inventor: Ronald P. Consiglio, Clermont, FL (US)

Correspondence Address: David M. Carter, Esq.;Carter, DeLuca, Farrell & Schmidt, LLP, Suite 225, 445 Broad Hollow Road, Melville, NY, 11747, US

(73) Assignee: DATAMAX CORPORATION

(21) Appl. No.: 10/962,117

(22) Filed: Oct. 8, 2004

(51) Int. Cl. C12Q 1/68

(52) U.S. Cl. 250/200

(57) ABSTRACT

A system and method are provided for detecting an adhesive label edge. The edge detection system employs two individually selectable light sources; one directed to providing transmissive mode detection while the second provides reflective mode detection of a label edge. The mode used is determined by the particular label media type used. The label edge detection system is dimensioned as either a subcomponent fabricated as part of any of a number of assemblies within a printer or as a stand-alone component positioned along a media path of the printer.

### BACKGROUND OF THE INVENTION

1. Field of the Invention

The present invention relates generally to thermal demand printing, and more particularly, to detection of print area edges in label printing.

2. Description of the Prior Art

Thermal demand printing has been in use for years where speed is required. Such applications include printing receipts, facsimiles and adhesive shipping labels. Generally, a thermal demand printer uses media coated with a thermal-reactive layer, which darkens with exposure to heat above a threshold temperature. The printer is equipped with a print head having a heating element configured to apply direct heat at or above the threshold temperature to an area of the thermal media, which is usually dimensioned as a roll of media. The threshold temperature is usually set at a temperature high enough not to be encountered by the media accidentally in a typical storage environment but below the flashpoint of the media, i.e., the point at which the media would combust.

In the specific case of adhesive label printing, the media used has a multitude of adhesive labels placed side-by-side on a substrate or backing and allows the labels to be readily peeled off individually. These labels come in a variety of sizes. The range of available label sizes and separation between labels gives rise to a significant printing issue.

The problem encountered with label printing involves correctly aligning the individual label for printing. Since the printing area is not continuous throughout the roll, i.e., the printing area is defined by the dimensions of the individual label and not by the dimensions of the media roll, the printer must detect when one label ends and the next one begins so that the printer is able to render the print within the bounds of the printable area of each label.

The criticality of this is best demonstrated by a hypothetical case wherein shipping labels are printed. In such a case, if the label is misaligned, the address may not be fully and clearly printed on the one label. In an automated shipping process, this inadequate shipping label may still end up on a package, but because the address is incomplete, the package cannot be efficiently delivered to its destination.

Therefore, a need exists for techniques for detecting an edge of each label on a media roll. Preferably, such a detection means should detect the label edge of commonly used label rolls, regardless of the presence, or lack thereof, of an edge indicator on the media.

### SUMMARY OF THE INVENTION

The present disclosure provides a system and method for detecting an edge of a label on a media roll. The system includes a first light source positioned along a media feed path and a photodetector positioned opposite the first light source so that the media feed path is positioned between the photodetector and the first light source. A second light source is positioned adjacent and on the same side of the media feed path as the photodetector.

Additionally, a controller is in electrical communication with the first and second light sources and the photodetector. The controller is configured for selectively activating the first light source and/or the second light source and for receiving feedback from the photodetector. A media feed mechanism is also provided for advancing media of a label roll, e.g., an adhesive label roll, along the media feed path. The media feed mechanism is controllable by the controller using the received feedback.

Further, the present disclosure provides a method for detecting a label edge. In the disclosed method, a first light source is positioned along a media feed path, with a photodetector positioned opposite the first light source, on either side of the media feed path. A second light source is positioned adjacent and on the same side of the media feed path as the photodetector. One of either the first light source or the second light source is selected for illuminating a media roll supporting the label as the media moves along a point on the media feed path. At least a portion of the illumination is received by the photodetector, wherein the portion has previously impacted the media. It is determined that an edge of the adhesive label has been encountered based on at least one characteristic of the illumination received by the photodetector.

### BRIEF DESCRIPTION OF THE DRAWINGS

These and other features, aspects, and advantages of the present disclosure will become better understood with regard to the following description, appended claims, and accompanying drawings wherein:

FIG. 1 is a schematic of an embodiment of a system for detecting an adhesive label edge in accordance with the present disclosure;

FIG. 2 is a flow chart of the steps performed by a method for detecting an adhesive label edge in accordance with the present disclosure;

FIGS. 3a and 3b illustrate two prior art label media types employed in accordance with the present disclosure; and

FIGS. 4 and 5 illustrate perspective views of a modular printer.

### DESCRIPTION OF THE PREFERRED EMBODIMENTS

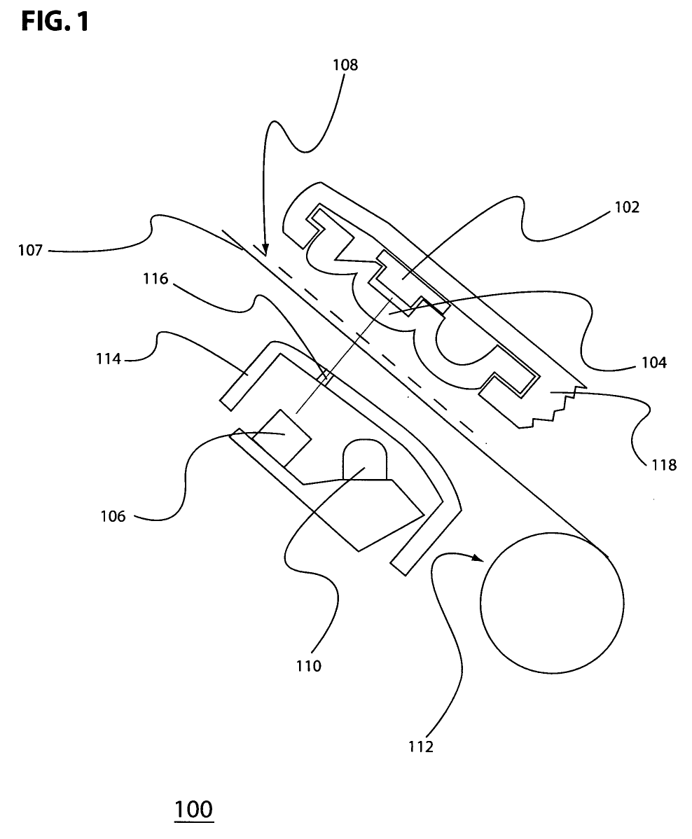

Referring to FIG. 1, an embodiment of the present disclosure provides a system 100 for detecting an adhesive label edge during printing. The system 100 includes a first light source 102 with a lens 104 designed to provide illumination as either a narrow spot or line on an adhesive label sheet 107 moving through a media feed path 108. The light source 102 may be an LED (light emitting diode) array arranged as a light bar. The lens may be a unitary body configured and dimensioned to slide within grooves of a support structure 118 of a thermal printer. The adhesive label sheet is fed through the media feed path 108 by a feed mechanism 112, which may incorporate a conveyor, manual or motorized rollers or sprockets to advance the sheet 107. A photodetector 106 is positioned across the media feed path 108 from the first light source 102 so that light transmitted through the media feed path 108 impacts the photodetector 106. As light passes through a portion of the media having a label, e.g., label sheet 107, the intensity of the transmitted light that impacts the photodetector 106 is diminished below a detection threshold, while light passing through portions of the media without a label impacts the photodetector 106 with intensity that is above the detection threshold. The system 100 may also incorporate a calibration component for calibrating the light output from the light source and the detection sensitivity of the photodetector as will be described below.

Additionally, a second light source 110 is positioned along side and at an angle to the photodetector 106. The second light source 110 is angled so as to illuminate a spot on the media backing of the label sheet as it moves through the media feed path 108 in a manner in which the light reflected by portions of the media impacts the photodetector 106. This reflection mode is appropriately used when the adhesive label roll separates individual labels with a black or absorptive marking or strip on a side of the media opposite the labels, e.g., the backing, thus light is reflected to the photodetector 106 by the underside of the labels but little or no light is reflected to the photodetector 106 by the portions of the media (or web as the label supporting media is known in the art) between the labels having the black or absorptive marking.

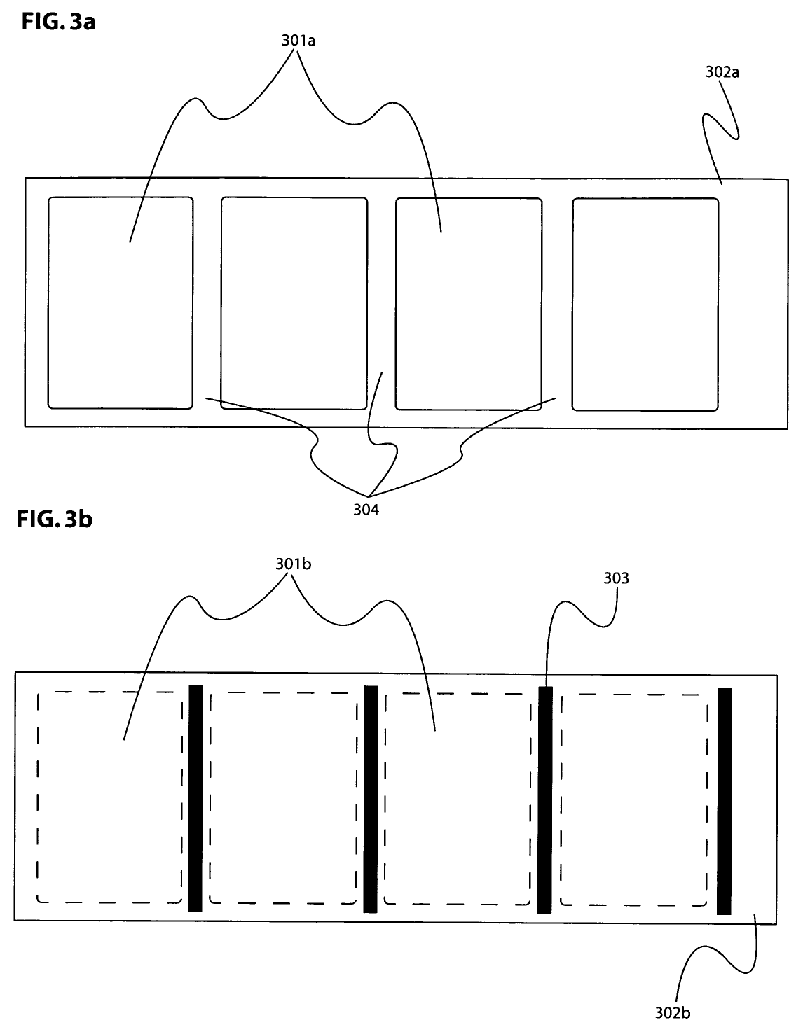

FIGS. 3a and 3b illustrate two types of label media. Referring to FIG. 3a, labels 301a are supported on a web 302a. The web may be formed from paper and have a waxy or non-adhesive film allowing the labels 301a to be easily peeled off of the web 302a. Each label 301a is spaced apart on the web 302a by a predetermined distance, forming a gap 304. The labels 301b in FIG. 3b are supported on a web 302b, as in FIG. 3a. However, the web 302b has a light absorpting strip 303 positioned between each label 301b, preferably on a side opposite the side supporting the labels 301b. Both types of label media are supported by the present disclosure.

An enclosing protective shell 114 having a slot aperture 116 formed thereon may be positioned to encase the photodetector 106 and second light source 110. The shell 114 provides two functions, the first being to protect the photodetector 106 and second light source 110 from damage, while also providing an increased sensitivity to smaller gap widths between labels by the use of the slot aperture 116 above the photodetector 106, which reduces the area of the focal plane normal to the relative motion of the gap between labels as it moves past the sensor.

The various components of the present embodiment, e.g., the first light source 102, second light source 110, and photodetector 106 are in electronic communication with a controller (not shown). The controller provides the activation inputs to the two light sources 102 and 110 and receives an output signal from the photodetector 106.

The controller also controls calibration components and the media feed mechanism using the output signal from the photodetector 106 as the control signal. The controller may analyze the control signal to determine the intensity of the light produced by the light sources 102 and 110, and increase or decrease the output power of the light sources, adjusting the edge detection sensitivity. For example, a typical calibration sequence may include removing media from the paper feed path, adjusting the light source (e.g., LED) current in steps from minimum to maximum and recording the sensor reading for each step; next, inserting the backing, i.e., with no label, into the feed path, adjusting LED current in steps from minimum to maximum and recording the sensor reading for each step; and lastly, inserting the backing with at least one label into the feed path, adjusting LED current in steps from minimum to maximum and recording the sensor reading for each step. The recorded data may then be used to determine a current setting for the LED that yields the best separation for label/backing to backing to media out conditions. Additionally, the data may be used to select edge detection thresholds based on the determined current setting. Alternatively, voltage levels of the light sources may be adjusted to calibrate the system.

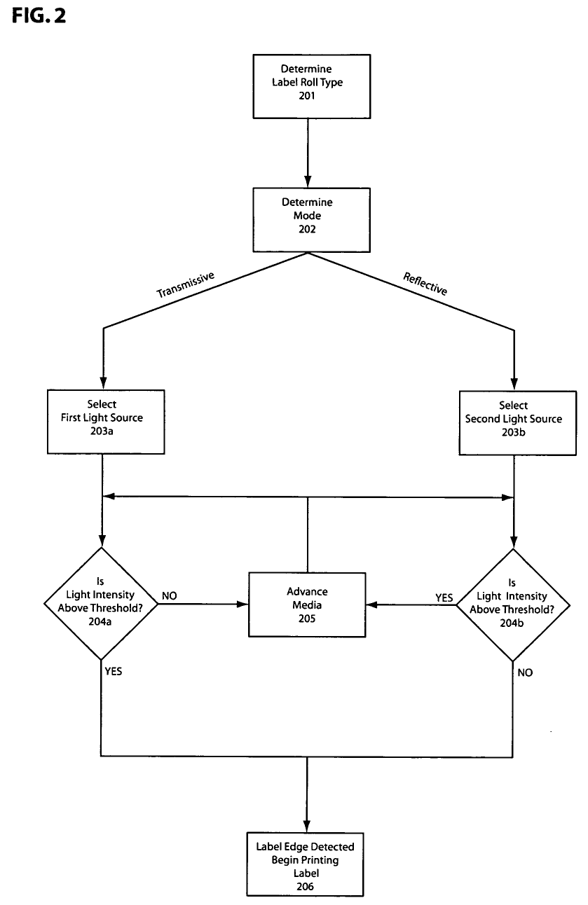

As shown in FIG. 2, the present embodiment provides a procedure for detecting adhesive label edges during printing. The detection procedure begins with a determination of the label roll type in step 201. The determination may be either manual or automatic in nature and directed towards identifying whether the label roll has an optically absorptive strip between each label or not. Upon completion of the roll type determination, step 202 provides for the selection of an appropriate operation mode, e.g., transmissive or reflective mode. The media roll type determines the mode selected: reflective mode is appropriate for use on rolls having the absorptive strip while transmissive mode is used on all other supported rolls. The selected light source is selected and activated in step 203a or 203b either in short duration pulses or continuously. If the operation mode is transmissive, the first light source 102 is selected in step 203a; in reflective mode, the second light source 110 is selected in step 203b. A photodetector 106 or other such device is used in step 204a or 204b to detect light emanating from the selected light source after having interacted with the media roll.

In transmissive mode light detection step 204a, the detected light intensity needs to be above a predetermined intensity threshold in order for the determination of edge detection to have occur at which point, printing begins in step 206; less then the threshold intensity value results in a ‘no edge detected’ determination and the media is advanced in step 205. Upon completion of step 205, the process loops back to step 204a until an edge is detected.

Alternatively in the reflective mode light detection step 204b, the detected light intensity needs to be below a predetermined intensity threshold in order for the determination of edge detection to have occur at which point, printing begins in step 206; detected light intensity greater then the threshold intensity value results in a ‘no edge detected’ determination and the media is advanced in step 205. Upon completion of step 205, the process loops back to step 204b until an edge is detected.

The present disclosure is dimensioned as a component installable, either during manufacture or as an after-market option, on commercially available label printers such as the modular printer disclosed in U.S. Pat. No. 6,616,362 issued on Sep. 9, 2003 and incorporated herein by reference in its entirety.

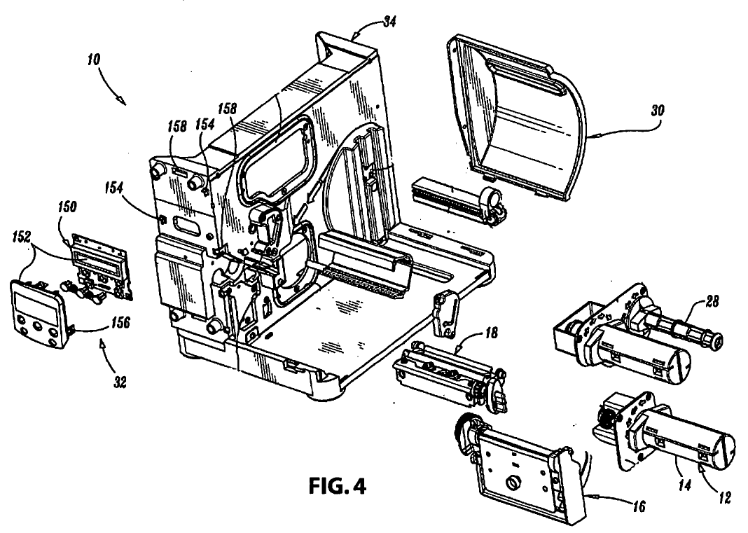

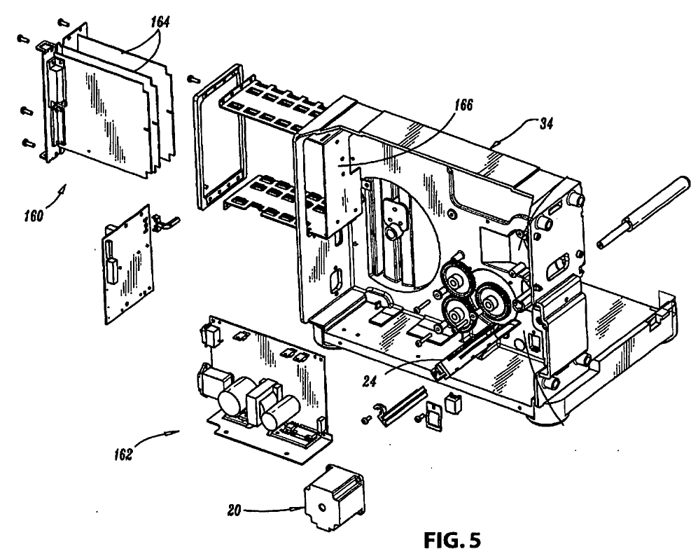

FIGS. 4 and 5 illustrate perspective views of a modular printer, with parts separated, shown generally as 10. More specifically, FIG. 4 illustrates the printing components of the modular printer and FIG. 5 illustrates the electrical and drive components of the modular printer.

Briefly, modular printer 10 includes a media take-up assembly 12 including a hub assembly 14 configured to support a media take-up roll (not shown), a support block assembly 16, a printhead assembly 18, a stepper motor assembly 20, a media sensor assembly 24 (see FIG. 5), a cover assembly 30 and a display assembly 32. The label edge detection system of the present disclosure as exemplified in FIG. 1, preferably, is a component part of the media sensor assembly 24, printhead assembly 18, or media take-up assembly 12, however the label edge detection system may also be dimensioned as a separate assembly positioned along a media path of the printer 10. When printer 10 is operated as a ribbon ink printer, a ribbon spool take-up assembly 28 may also be provided in conjunction with the media take-up assembly. Each of the above-identified assemblies is removably supported on a support body 34. The support body defines an internal support wall of the modular printer and is configured to properly align each of the assemblies with respect to each of the other assemblies within the printer. Support body 34 is preferably formed from a heat conductive material, such as an aluminum support body, to facilitate the removal of heat from printer 10. However, other materials may also be used to form housing 34 including ceramics, plastics, sheet metal etc.

As discussed above, printer 10 has a display assembly 32. Display assembly 32 includes a module 150 having an LED display and a casing 152. Module 150 is positioned between diametrically opposed guide brackets 154 formed on support body 34. Opposite corners of module 150 are subsequently secured to support body 34 by screws. Casing 152 includes a plurality of flexible brackets 156, which can be snap fit to support body 34 over module 150. Support body 34 includes receiving structure 158 formed therein. Alternately, other known fastening devices may be used to secure module 150 and casing 152 to support body 34.

Referring again to FIG. 5, the electrical and drive components of the ink printer 10 are secured to the opposite side of support body 34 than are the printing components of the printer 10. As discussed above, stepper motor assembly 20 is secured to support body 34 on the side opposite the printing components. Electronic circuitry 160 and electric drive assembly 162 to operate printer 10 are secured to the support body 34 on the side opposite the printing components. Electronic circuitry 160 is in the form of circuit boards 164, which can be installed in printer 10 by sliding the circuit boards through an opening 166 formed in support body 34. The circuit boards can be chosen to suit the particular printing operation to be performed. For example, the circuitry 160 can be changed for different communications interfaces. Alternatively, software can be downloaded via a communication port to control a particular printing application.

The described embodiments of the present disclosure are intended to be illustrative rather than restrictive, and are not intended to represent every embodiment of the present disclosure. Various modifications and variations can be made without departing from the spirit or scope of the disclosure as set forth in the following claims both literally and in equivalents recognized in law.

What is claimed is:

1. A system for detecting an edge of a label of a media roll including a plurality of labels, said system comprising:
a first light source positioned along a media feed path;
a photodetector positioned opposite said first light source and with said media feed path positioned between said photodetector and said first light source;
a second light source positioned adjacent and on the same side of said media feed path as said photodetector;
a controller in electrical communication with said first and second light source and said photodetector, said controller being configured for selectively activating said first light source and/or said second light source and for receiving feedback from said photodetector.

2. The system of claim 1, further comprising a media feed mechanism for advancing the media roll along said media feed path.

3. The system of claim 2, wherein said media feed mechanism is a motorized roller assembly controllable by said controller using said received feedback.

4. The system of claim 2, wherein said media feed mechanism is manually operated roller assembly controllable by an operator.

5. The system of claim 1, wherein said first light source is aligned to illuminate a surface of said media such that a portion of said illumination is transmitted through said media and impacts said photodetector.

6. The system of claim 5, wherein said transmitted light varies in intensity depending on whether said transmitted light traveled through a portion of said label or said media, said illumination is substantially absorbed by said labels reducing said portion of transmitted light impacting said photodetector to an intensity below a detection threshold of said photodetector.

7. The system of claim 1, wherein said second light source is aligned to illuminate a surface of said media such that a reflected beam results, which intersects with said photodetector.

8. The system of claim 7, wherein an optically absorptive strip is imprinted on said media and positioned between said labels.

9. The system of claim 1, further comprising a calibration component for calibrating said first and second light source, wherein said calibration components adjusts voltage and/or current to said selected light source.

10. A method for detecting a label edge, said method comprising the steps of:
providing a first light source positioned along a media feed path;
providing a photodetector positioned opposite said first light source and with said media feed path positioned between said photodetector and said first light source;
providing a second light source positioned adjacent and on the same side of said media feed path as said photodetector;
selecting one of either said first light source or said second light source;
illuminating a media roll supporting said label with said selected light source as said media moves along a point on said media feed path;
receiving at least a portion of said illumination with said photodetector, wherein said portion has previously impacted said media; and
determining whether an edge of said adhesive label has been encountered based on at least one characteristic of said illumination received by said photodetector.

11. The method of claim 10, wherein said first light source is aligned to illuminate a surface of said media such that a portion of said illumination is transmitted through said media and impacts said photodetector.

12. The method of claim 11, wherein said transmitted light varies in intensity depending on whether said transmitted light traveled through a portion of said label or said media, said illumination is substantially absorbed by said adhesive labels reducing said portion of transmitted light impacting said photodetector to an intensity below a detection threshold of said photodetector.

13. The method of claim 10, wherein said second light source is aligned to illuminate a surface of said media such that a reflected beam results, which intersects with said photodetector.

14. The method of claim 13, wherein an optically absorptive strip is imprinted on said media and positioned between said labels.

15. The method of claim 10, further comprising the step of calibrating said selected light source by adjusting voltage and/or current to said selected light source.

16. A system for detecting an edge of a label of a media roll, said system comprising:
means for providing a first light source positioned along a media feed path;
means for detecting light positioned opposite said first light source and with said media feed path positioned between said light detecting means and said first light source means;
means for providing a second light source positioned adjacent and on the same side of said media feed path as said light detecting means;
means for selecting one of either said first light source or said second light source means; and
means for determining whether an edge of said label has been encountered based on at least one characteristic of said light detected by said light detecting means.

17. The system of claim 16, further comprising a means for calibrating said selected light source means, wherein said calibration means adjusts voltage and/or current to said selected light source means.

18. The system of claim 16, further comprising a means for advancing said media roll through said media feed path.

19. The system of claim 16, wherein said first light source means is aligned to illuminate a surface of said media such that a portion of said illumination is transmitted through said media and impacts said light detecting means.

20. The system of claim 19, wherein said transmitted light varies in intensity depending on whether said transmitted light traveled through a portion of said label or said media, said illumination is substantially absorbed by said label reducing said portion of transmitted light impacting said light detecting means to an intensity below a detection threshold of said light detecting means.

21. The system of claim 16, wherein said second light source means is aligned to illuminate a surface of said media such that a reflected beam results, which intersects with said light detecting means.

22. The system of claim 21, wherein an optically absorptive strip is imprinted on said media and positioned between said labels.
